(19) United States
(12) Patent Application Publication
(10) Pub. No.: US 2015/0253316 A1
(43) Pub. Date: Sep. 10, 2015

(54) MEMBRANE SUPPORT FOR DETECTING SUGAR CHAIN-RECOGNIZING MOLECULE

(71) Applicant: SUMITOMO BAKELITE CO., LTD., Tokyo (JP)

(72) Inventor: Masaaki Toyota, Shinagawa-ku (JP)

(73) Assignee: SUMITOMO BAKELITE CO., LTD., Shinagawa-ku, Tokyo (JP)

(21) Appl. No.: 14/437,696

(22) PCT Filed: Oct. 30, 2013
(86) PCT No.: PCT/JP13/79369

§ 371 Date: Apr. 22, 2015

(30) Foreign Application Priority Data
Oct. 31, 2012 (JP) 2012-240254

(51) Int. Cl. G01N 33/543; G01N 33/544

(52) U.S. Cl. CPC G01N 33/54306; G01N 33/544; G01N 2405/10; G01N 2333/4703

(57) ABSTRACT

Provided are substance and method enabling quick and easy detection of a sugar chain-recognizing molecule with high sensitivity in any place. Specifically, provided are: a membrane support for immunochromatography, the membrane support comprising a detection line on which a protein having the sugar chain binding thereto is immobilized; a device comprising the membrane support; and a method using these for detecting by immunochromatography a molecule capable of recognizing the sugar chain.

### TECHNICAL FIELD

The present invention relates to a membrane support for detecting a molecule capable of recognizing a sugar chain, a device for detecting by immunochromatography a molecule capable of recognizing a sugar chain, and a method for detecting by immunochromatography a molecule capable of recognizing a sugar chain.

### BACKGROUND ART

Cases are known in which an autoantibody recognizing a sugar chain moiety of a glycolipid is detected in a serum of a patient with an inflammatory disease or an autoimmune disease. Particularly, it has been known that a large number of glycolipids are present in nervous tissues. Such an autoantibody as mentioned above is known to cause an autoimmune nerve disease, damaging nerves.

Against the diseases, treatments are performed such as plasmapheresis and immunoadsorption therapy for removing an autoantibody from the body of a patient, and intravenous injection therapy with a large amount of immunoglobulins that neutralize an autoantibody. However, since these treatments are effective for approximately one week from the onset, the diagnosis result has to be provided quickly.

The diagnosis method for autoimmune diseases and the like includes an ELISA method using a microplate on which a glycolipid itself is immobilized (PTL 1). Nevertheless, this diagnosis method requires an analyzer such as a microplate reader, involves the complicated step for detecting an autoantibody or the like, and further requires specialized knowledge for the analysis of the obtained result. For these reasons, samples collected from a patient or the like has to be sent to an examination center for the examination. Hence, the ELISA method that requires a lot of time for the diagnosis is less effective against autoimmune diseases and the like against which the diagnosis result has to be provided quickly. Additionally, a molecule capable of recognizing a sugar chain (sugar chain-recognizing molecule) generally binds weakly to the sugar chain. Hence, as described in Examples later, the conventional system for detecting a sugar chain-recognizing molecule has a low sensitivity.

Accordingly, in the diagnosis and so forth of autoimmune diseases and the like, it has been desired to develop methods capable of detecting a sugar chain-recognizing molecule such as an autoantibody quickly and easily with high sensitivity in any place. However, such methods are not put into practical use yet at present.

### CITATION LIST

### Patent Literature

[PTL 1] Japanese Unexamined Patent Application Publication No. 2003-294752

### SUMMARY OF INVENTION

### Technical Problem

The present invention has been made in view of the above-described problems of the conventional technique. An object of the present invention is to provide substance and method enabling quick and easy detection of a sugar chain-recognizing molecule with high sensitivity in any place.

### Solution to Problem

Immunochromatography is known as a method capable of detecting a particular molecule quickly and easily in any place. Nonetheless, generally, a sugar chain-recognizing molecule weakly binds to a sugar chain, and it has been difficult to immobilize a sugar chain highly densely on a membrane support used in immunochromatography. Hence, a sufficient sensitivity for detecting a sugar chain-recognizing molecule has not been successfully obtained in immunochromatography.

Nevertheless, as a result of earnestly studied, the present inventors have revealed that when a protein having a sugar chain binding thereto is used as a probe for detecting a sugar chain-recognizing molecule, the sugar chain can be highly densely immobilized on a membrane support used in immunochromatography. Further, it has been found out that immunochromatography using a membrane support on which such a sugar chain-binding protein is immobilized is capable of detecting a sugar chain-recognizing molecule with high sensitivity in comparison with a conventional case where a glycolipid is immobilized. This discovery has led to the completion of the present invention. Specifically, the present invention provides the following.
<1> A membrane support for use in a device for detecting by immunochromatography a molecule capable of recognizing a sugar chain, the membrane support comprising a detection line on which a protein having the sugar chain binding thereto is immobilized.
<2> The membrane support according to <1>, wherein the protein having the sugar chain binding thereto is obtained by binding a reducing terminal of the sugar chain to an amino group of the protein by a reductive amination reaction.
<3> The membrane support according to any one of <1> and <2>, wherein the protein is albumin.
<4> A method for detecting by immunochromatography a molecule capable of recognizing a sugar chain, the method comprising:

bringing a test sample into contact with a molecule which is labeled and recognizes a molecule capable of recognizing a sugar chain;

then developing the test sample toward the detection line of the membrane support according to any one of <1> to <3>; and

detecting the labeled substance on the detection line.
<5> A device for detecting by immunochromatography a molecule capable of recognizing a sugar chain, the device comprising:

the membrane support according to any one of <1> to <3>; and

at least one component selected from the group consisting of the following (a) to (c):

(a) a sample pad;

(b) a conjugate pad; and

(c) an absorbent pad.

### Advantageous Effects of Invention

The present invention makes it possible to detect a sugar chain-recognizing molecule quickly and easily with high sensitivity in any place.

### BRIEF DESCRIPTION OF DRAWINGS

FIG. 1 is a schematic plan view showing a preferred embodiment of a device for detecting by immunochromatography a molecule capable of recognizing a sugar chain of the present invention.

FIG. 2 is a schematic side cross-sectional view showing a cut surface obtained by cutting the device shown in FIG. 1 along the line I-II in FIG. 1.

FIG. 3 is a photograph for illustrating the result of staining, with a protein staining reagent, gels on which albumin and albumin to which a sugar chain (GM1) was bound (GM1-binding albumin) were developed by SDS-PAGE. Five lanes of the GM1-binding albumin respectively show the results of developing the reaction products between 66 μg of the albumin and 1000 μg, 500 μg, 250 μg, 125 μg, and 62.5 μg of GM1 in this order from the left.

FIG. 4 is a photograph for illustrating the result of transferring to PVDF membranes the gels on which the albumin and the GM1-binding albumin were developed by SDS-PAGE, allowing HRP-labeled cholera toxin β subunit (HRP-CTB) to act on the membranes, and staining the gels using 4-chloro-1-naphthol as a chromogenic substrate. Five lanes of the GM1-binding albumin respectively show the results of developing the reaction products between 66 μg of the albumin and 1000 μg, 500 μg, 250 μg, 125 μg, and 62.5 μg of GM1 in this order from the left.

FIG. 5 is a photograph for illustrating the result of allowing HRP-CTB to act on membrane supports (HiFlow Plus HFB180UBCAST, HiFlow Plus HFB07502, HiFlow Plus HFB12002) on which the GM1-binding albumin and the albumin were immobilized, and staining the membrane supports using 4-chloro-1-naphthol as a chromogenic substrate.

FIG. 6 is a photograph for illustrating the result of allowing HRP-CTB to act on the membrane supports on which the GM1-binding albumin and the albumin were immobilized, and staining the membrane supports using 4-chloro-1-naphthol as a chromogenic substrate. In the figure, the quantities (100 ng, 10 ng, 1 ng) indicate the amount of the GM1-binding albumin or the albumin spotted on the membrane support.

FIG. 7 is a photograph for illustrating the result of allowing HRP-CTB to act on membrane supports on which the GM1-binding albumin and GM1 ganglioside were immobilized, and staining the membrane supports using 4-chloro-1-naphthol as a chromogenic substrate. In the figure, the quantities (100 ng, 10 ng) indicate the amount of the GM1-binding albumin or GM1 ganglioside spotted on the membrane support.

FIG. 8 is a photograph for illustrating the result of allowing HRP-CTB to act on membrane supports on which the GM1-binding albumin and GM1 ganglioside were immobilized, and staining the membrane supports using 4-chloro-1-naphthol as a chromogenic substrate. In the figure, concentrations (1.0 μg/mL, 0.1 μg/mL, 0.01 μg/mL) indicate the concentrations of the HRP-CTB allowed to act on the membrane supports.

### DESCRIPTION OF EMBODIMENTS

(Membrane Support for Immunochromatography)

The present invention provides a membrane support for use in a device for detecting by immunochromatography a molecule capable of recognizing a sugar chain, the membrane support comprising a detection line on which a protein having the sugar chain binding thereto is immobilized.

In the present invention, the term “sugar chain” refers to a polysaccharide in which two or more monosaccharides (monosaccharide and a derivative thereof) are linked by a glycosidic bond, and which is a molecule capable of specifically binding to a sugar chain-recognizing molecule to be detected by the immunochromatography according to the present invention. Moreover, such a sugar chain may be constituted of one type of monosaccharides (homogeneous sugar chain), or may be constituted of two or more types of monosaccharides (heterogeneous sugar chain). The type and the number of monosaccharides constituting the sugar chain are not particularly limited. Additionally, the glycosidic bond between monosaccharides maybe either an α-bond or a β-bond. Further, the sugar chain maybe a sugar chain derived from nature, or one obtained by artificially modifying a sugar chain derived from nature through addition, substitution, or the like of a functional group, or a chemically synthesized sugar chain.

In the present invention, the “molecule capable of recognizing a sugar chain” is not particularly limited. Examples thereof include antibodies capable of recognizing the sugar chain, and proteins capable of recognizing the sugar chain (lectins).

In the present invention, regarding the “protein” having the sugar chain binding thereto, the type and the number of amino acids constituting the protein are not particularly limited, as long as the amino acids are capable of multivalent binding to the sugar chain. Moreover, the “protein” may be one obtained from natural products such as plants, animals, or microorganisms (such as bacteria and viruses) by isolation/extraction/purification, or may be one synthesized by a genetic approach using a cell-free protein synthesis system (for example, a reticulocyte extract, wheat germ extract), Escherichia coli, animal cells, insect cells, plant cells, or the like. Further, the “protein” may be chemically synthesized using an automated synthesizer or the like. The “protein” according to the present invention would not have a sugar chain unless artificially modified with the sugar chain. Accordingly, from the viewpoint that a larger amount of the sugar chain according to the present invention is likely to bind, albumin and ribonuclease A are preferable as natural products. A protein synthesized by a genetic approach using Escherichia coli and a chemically synthesized protein are also preferable from the same viewpoint.

In the present invention, the method for preparing the protein having the sugar chain binding thereto is not particularly limited. Examples thereof include a method by which a reducing terminal of the sugar chain is bound to an amino group of the protein (reductive amination reaction); a method by which a reducing terminal of the sugar chain is bromo-acetylated and covalently bonded to a thiol group in cysteine of the protein; a method by which an amino group is introduced to a reducing terminal of the sugar chain and covalently bonded to a carboxyl group of the protein; and a method by which amino acids having the sugar chain chemically binding thereto are linked to each other by a polymerization reaction to synthesize the protein. A reductive amination reaction is preferable from the viewpoints that it is not necessary to newly introduce a functional group to a reducing terminal of the sugar chain and that the reaction is simple.

Moreover, although depending on the types of the sugar chain and the protein, the aforementioned method for preparing the protein having the sugar chain binding thereto, and so forth, the average number of the sugar chains binding per molecule of the protein is normally 1 to 40, and preferably 5 to 25 from the viewpoints of higher density of the sugar chain on the protein and the easiness of preparing the protein having the sugar chain binding thereto.

In the present invention, the “membrane support” on which the sugar chain-binding protein is immobilized should be a membrane made of a material capable of immobilizing the sugar chain-binding protein, and also capable of developing by capillary action a test sample subjected to the immunochromatography. Examples of such a material include celluloses such as nitrocellulose, polyvinylidene fluoride (PVDF), polyethersulfone (PES), polyethylene terephthalate (PET), polyethylenes, polypropylenes, nylons, and glass fibers. Moreover, the pore diameter of the membrane support of the present invention is not particularly limited, and should be adjusted as appropriate by taking the developing speed of a test sample and so forth into consideration. For example, the pore diameter is 0.1 to 10 μm.

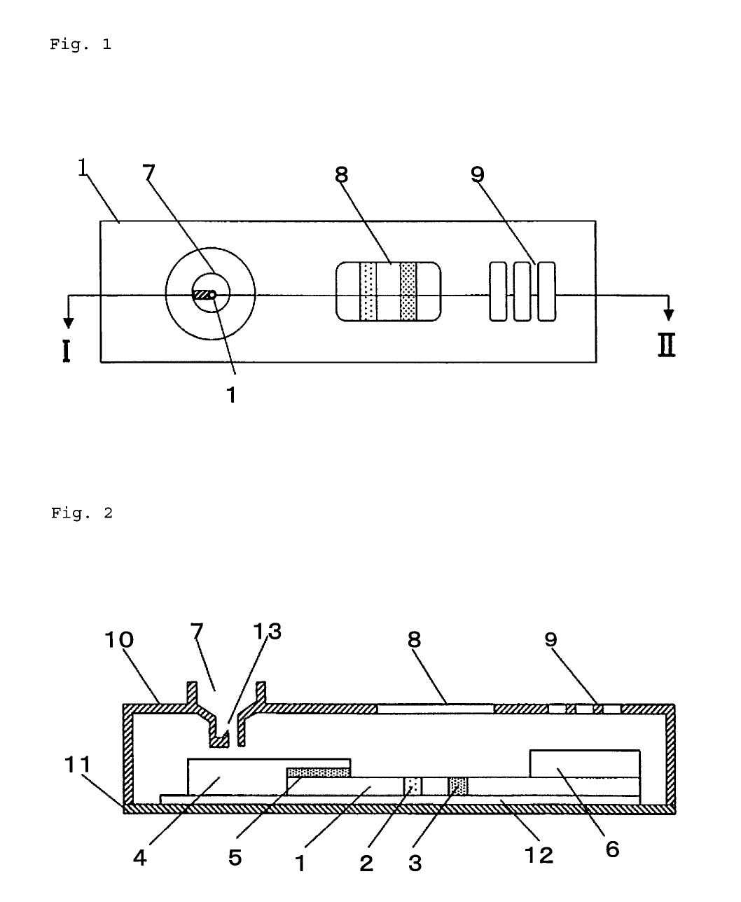

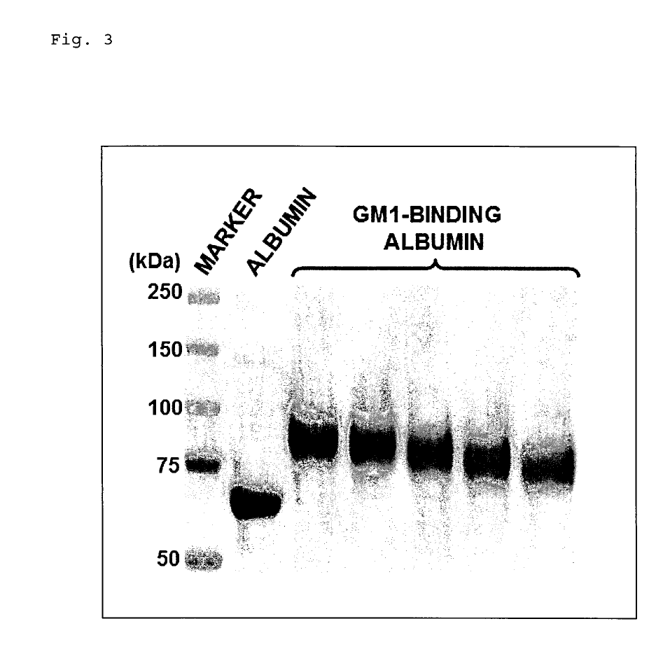

The “detection line” of the membrane support is a site for capturing and detecting a sugar chain-recognizing molecule in a test sample developed on the membrane support. Thus, the “detection line” needs to be disposed at a downstream side in the membrane support. Note that, in the present invention, the terms upstream side and downstream side respectively mean an upstream side (left side in FIGS. 1 and 2 to be described later) and a downstream side (right side in FIGS. 1 and 2 to be described later) in a flow in which a test sample is developed on the membrane support.

Moreover, the “detection line” needs to have a sugar chain-binding protein immobilized thereon, the protein being a molecule for capturing a sugar chain-recognizing molecule. The immobilization method is not particularly limited. Examples thereof include methods utilizing physical adsorption, electrostatic interaction, hydrophobic interaction, or a crosslinker. Further, the amount of the sugar chain-binding protein immobilized should be adjusted as appropriate in accordance with the material of the membrane support, the binding to the sugar chain-recognizing molecule, the type of a label of a molecule which recognizes a molecule capable of recognizing a sugar chain to be described later, and so forth. The amount is for example 1 ng to 1 μg.

(Method for Detecting by Immunochromatography Molecule Capable of Recognizing Sugar Chain)

The present invent ion provides a method for detecting by immunochromatography a molecule capable of recognizing a sugar chain, the method comprising:

bringing a test sample into contact with a molecule which is labeled and recognizes a molecule capable of recognizing a sugar chain;

then developing the test sample toward the detection line of the membrane support of the present invention; and

detecting the labeled substance on the detection line.

In the present invention, the “test sample” is not particularly limited. Examples thereof includes blood, serum, plasma, cerebrospinal fluid, saliva, sputum, tear, eye mucus, mucous membranes of the oral cavity and the nasal cavity, urine, fecal matter, skin, various organs, muscles, bones, and nerves, which are isolated from bacterial cells, soil, or animals such as human, as well as liquid extracts from these samples.

In the present invention, the “molecule which recognizes a molecule capable of recognizing a sugar chain” is not particularly limited, and may be an antibody capable of recognizing the sugar chain-recognizing molecule, or may be Protein A or Protein G in a case where the sugar chain-recognizing molecule is an antibody. Further, the molecule may be a sugar chain recognized by the sugar chain-recognizing molecule. Meanwhile, examples of a labeling substance added to such a “molecule which recognizes a molecule capable of recognizing a sugar chain” include a color labeling substance and an enzyme labeling substance. Examples of the color labeling substance include, besides colloidal metals such as colloidal gold and colloidal platinum, latexes such as natural rubber latexes and synthetic latexes including polystyrene latex colored with red, blue, and other pigments. Examples of the enzyme labeling substance include horseradish peroxidase (HRP), β-galactosidase, luciferases, and alkaline phosphatases.

The test sample and the molecule which is labeled and recognizes a molecule capable of recognizing a sugar chain should be “brought into contact with” each other until the test sample introduced to the membrane support reaches the detection line. For example, the molecule which is labeled and recognizes a molecule capable of recognizing a sugar chain may be added to the sample so that the two may be brought into contact with each other, before the test sample is introduced to the membrane support of the present invention or the device of the present invention to be described later. Alternatively, the contact may be achieved by passing the test sample through the inside of a conjugate pad to be described later.

In the present invention, in order to efficiently develop the test sample and the molecule which is labeled and recognizes a molecule capable of recognizing a sugar chain by capillary action, an eluent maybe used. Examples of the eluent include buffers containing at least one among proteins, polysaccharides, surfactants, organic solvents, polymers, and the like. The eluent may be mixed with the test sample and then introduced to the membrane support. Alternatively, after the test sample and so on are introduced to the membrane support, the eluent may be introduced to the membrane support from the upstream side thereof.

After that, if the test sample contains a sugar chain-recognizing molecule, a complex formed by the contact between the sugar chain-recognizing molecule and the molecule which is labeled and recognizes the molecule is captured by a sugar chain on the detection line. Thereby, the labeled substance accumulates on the detection line.

Thus, the present invention makes it possible to judge the presence or absence of a sugar chain-recognizing molecule in a test sample by detecting a labeled substance on the detection line. If the labeling substance is the color labeling substance, “detecting a labeled substance” can be carried out by visually observing a color developed according to the accumulation of the labeled substance on the detection line. Meanwhile, if the labeling substance is the enzyme labeling substance, “detecting a labeled substance” can be carried out by visually observing coloring formed by adding a chromogenic substrate of the enzyme to the detection line. In the case where HRP is used as the enzyme labeling substance, examples of such a chromogenic substrate include 4-chloro-1-naphthol, ABTS, O-phenylenediamine (OPD), and tetramethylbenzidine (TMB).

(Device for Immunochromatography)

The present invention provides a device for detecting by immunochromatography a molecule capable of recognizing a sugar chain, the device comprising:

the above-described membrane support of the present invention; and

at least one component selected from the group consisting of the following (a) to (c):

(a) a sample pad;

(b) a conjugate pad; and

(c) an absorbent pad. FIGS. 1 and 2 show a preferred example of such a device of the present invention. FIG. 1 is a schematic plan view of the device. FIG. 2 is a schematic side cross-sectional view showing a cut surface obtained by cutting the device shown in FIG. 1 along the line I-II in FIG. 1. Hereinafter, the device of the present invention will be described with reference to these drawings.

The device of the present invention is a device comprising: the membrane support 1 of the present invention; and at least one component selected from the group consisting of a sample pad 4, a conjugate pad 5, and an absorbent pad 6, as shown in FIG. 2.

The membrane support 1 is as described above, but may comprise, in addition to the above-described detection line 2, a control line 3 as shown in FIGS. 1 and 2.

The control line 3 is a portion for capturing the above-described molecule which is labeled and recognizes a molecule capable of recognizing a sugar chain. Accordingly, in a case where the labeled substance is not detected on the detection line 2 but is detected only on the control line 3, it is possible to determine that no sugar chain-recognizing molecule is present in the test sample. Further, a molecule (for example, antibody, lectin, aptamer) capable of specifically binding to the molecule which is labeled and recognizes a molecule capable of recognizing a sugar chain is immobilized on the control line 3 to capture the above-described molecule.

The sample pad 4 is a portion for retaining and passing therethrough a test sample, an eluent, and so on introduced to the device of the present invention. Thus, in the device of the present invention, the sample pad 4 needs to be disposed in direct or indirect contact with the membrane support 1, and on the upstream side of the membrane support 1.

The conjugate pad 5 is configured to support the above-described molecule which is labeled and recognizes a molecule capable of recognizing a sugar chain, and is a portion for bring the molecule and a test sample into contact with each other. Thus, in the device of the present invention, the conjugate pad 5 needs to be disposed in direct or indirect contact with the sample pad 4 and the membrane support 1, and on the downstream side of the sample pad 4 and the upstream side of the membrane support 1.

The absorbent pad 6 is a portion for promoting a development of a test sample on the membrane support and inhibiting a countercurrent, by absorbing excessive test sample, eluent, and so on having passed through the membrane support. Thus, in the device of the present invention, the absorbent pad 6 needs to be disposed in direct or indirect contact with the membrane support 1, and on the downstream side of the membrane support 1.

The material of the sample pad 4, the conjugate pad 5, and the absorbent pad 6 is not particularly limited. Examples thereof include celluloses such as nitrocellulose, polyvinylidene fluoride (PVDF), polyethersulfone (PES), polyethylene terephthalate (PET), polyethylenes, polypropylenes, nylons, and glass fibers.

The device of the present invention may comprise a retaining plate 12, as shown in FIGS. 1 and 2, for retaining the above-described membrane support 1, sample pad 4, conjugate pad 5, or absorbent pad 6. The material of the retaining plate 12 is preferably a plastic from the points of easiness of formation, cost, and weight reduction. Various plastic materials can be selected as the material of the plastic, and the selection is made as appropriate in accordance with the use purpose, treatment, solvent and physiologically active substance to be used, and characteristics of the detection method, by taking the formability, heat resistance, chemical resistance, adsorption, and so forth into consideration. Examples thereof include polystyrenes, polyethylenes, polyvinyl chlorides, polypropylenes, polycarbonates, polyesters, polymethyl methacrylates, polyvinyl acetates, vinyl-acetate copolymers, styrene-methylmethacrylate copolymers, acrylonitrile-styrene copolymers, acrylonitrile-butadiene-styrene copolymers, nylons, polymethylpentenes, silicone resins, amino resins, polysulfones, polyethersulfones, polyetherimides, fluorinated resins, polyimides, and the like. Moreover, these plastic materials may be mixed as appropriate with an additive such as a pigment, a dye, an antioxidant, or a flame retardant.

Further, the device of the present invention may comprise a casing (an upper casing 10 and a lower casing 11) for fixing and storing the above-described membrane support 1, pads, and retaining plate 12.

The matrix of the upper casing 10 and the lower casing 11 is preferably a plastic from the points of easiness of formation, cost, and weight reduction. Examples of the material of the plastic include polystyrenes, polyethylenes, polyvinyl chlorides, polypropylenes, polycarbonates, polyesters, polymethyl methacrylates, polyvinyl acetates, vinyl-acetate copolymers, styrene-methylmethacrylate copolymers, acrylonitrile-styrene copolymers, acrylonitrile-butadiene-styrene copolymers, nylons, polymethylpentenes, silicone resins, amino resins, polysulfones, polyethersulfones, polyetherimides, fluorinated resins, polyimides, and the like. Moreover, these plastic materials may be mixed as appropriate with an additive such as a pigment, a dye, an antioxidant, or a flame retardant.

Further, the upper casing 10 and the lower casing 11 may comprise a structure formed as appropriate to fix the contents such as the sample pad 4, the conjugate pad 5, the membrane support 1, the absorbent pad 6, and the retaining plate 12.

In addition, in the device of the present invention, the upper casing 10 may comprise an introduction port 7, a determination window 8, or an air hole 9 formed as shown in FIGS. 1 and 2.

The introduction port 7 is a portion for introducing a test sample to be subjected to the immunochromatography. The shape of the introduction port 7 is not particularly limited. The introduction port 7 may have a protrusion 13 therein as shown in FIGS. 1 and 2. This protrusion breaks a seal of a container housing a test sample (container for collecting a specimen), making it possible to develop the specimen quickly toward the detection line 2 without leaking the specimen to the outside (as to the container for collecting a specimen, the seal, and the protrusion, see Japanese Patent Application Publication No. 2010-38797).

The determination window 8 is a portion for visually recognizing the detection line 2 and the control line 3 to be described later of the membrane support 1 to, for example, determine the presence or absence of a sugar chain-recognizing molecule in a test sample. The determination window 8 may be formed, for example, by perforating the upper casing in a thickness direction thereof, and further transparent plastic plate or film may be added to the through hole.

The air hole 9 is a portion for discharging air inside the casing. Thereby, the efficiency of absorbing a test sample by the absorbent pad 6 to be described later is improved, consequently enabling a quick development of the test sample on the membrane support. The air hole 9 should be formed by perforating the upper casing in the thickness direction. The shape and the number of the air hole 9 are not particularly limited. An example thereof includes three through holes having a rectangular shape as shown in FIGS. 1 and 2.

The size of the device of the present invention is not particularly limited. For example, the device (casings) shown in FIGS. 1 and 2 has the following dimensions: a length of 5 to 11 cm, a width of 1 to 3 cm, and a height of 0.2 to 1.5 cm.

Hereinabove, the preferred embodiment of the device of the present invention has been described. However, the device of the present invention is not limited to the above-described embodiment. Those skilled in the art can apply a modification, alteration, addition, or the like to the component, material, structure, and so forth of the device as appropriate while taking the above into consideration.

### EXAMPLES

Hereinafter, the present invention will be more specifically described on the basis of Examples and Comparative Examples. However, the present invention is not limited to the following Examples.

<Preparation of Protein Having Sugar Chain Binding Thereto>

First, in order to bind a reducing terminal of a sugar chain (GM1) of a glycolipid (ganglioside GM1) to an amino group of human-derived albumin, a reductive amination reaction was carried out. In this reductive amination reaction, the amount of the sugar chain subjected to the reaction was set at 1000 μg, 500 μg, 250 μg, 125 μg, or 62.5 μg, relative to 66 μg of the albumin.

Next, the obtained product or the albumin was developed alone by SDS-PAGE, and stained with a protein staining reagent (GelCode(registered trademark) blue staining reagent, manufactured by Thermo Fisher Scientific Inc.). FIG. 3 shows the obtained result.

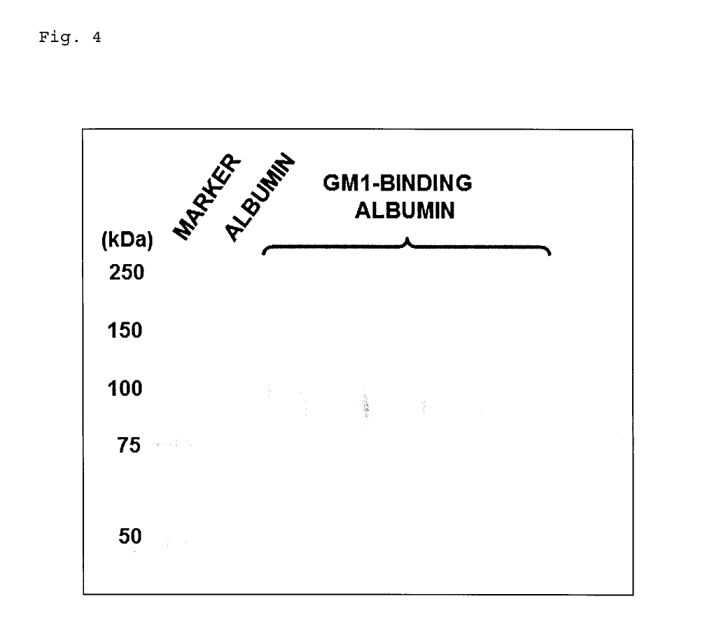

Moreover, the gel on which the product or the albumin was developed alone was transferred to a PVDF membrane. Then, cholera toxin β subunit known as a GM1-recognizing molecule was labeled with HRP. The resultant (hereinafter may also be referred to as “HRP-CTB”. The HRP-CTB concentration: 0.2 μg/mL) was allowed to act on the PVDF membrane. Subsequently, the gel was stained with an HRP chromogenic substrate 4-chloro-1-naphthol. FIG. 4 shows the obtained result.

As apparent from the result shown in FIG. 4, in the lane where only the albumin was electrophoresed, no coloring by HRP-CTB was observed. On the other hand, in the lanes where the prepared five GM1-binding albumins were migrated, bands of approximately 86, 84, 79, 76, and 73 kDa were specifically detected from the coloring by the HRP-CTB. Thus, it was revealed that it was possible to prepare by the reductive amination reaction a glycoprotein having GM1 binding to albumin.

Moreover, as shown in FIG. 3, as the result of staining using the GelCode blue staining reagent that uniformly stained a protein, a band of approximately 66 kDa that corresponded to the molecular weight of the albumin was observed in the lane where the albumin was electrophoresed. Further, since the molecular weight of the prepared GM1-binding albumins was approximately 86, 84, 79, 76, or 73 kDa as shown in FIG. 4, it was found that the increase in the molecular weight due to the sugar chain bound to the albumin was approximately 20, 18, 13, 10, or 7 kDa. In addition, since the molecular weight per GM1 sugar chain is approximately 1 kDa, it was revealed that the average number of the sugar chain binding to one molecule of the albumin was approximately 20, 18, 13, 10, or 7. Furthermore, in consideration of the amount of the sugar chain thus reacted, it was also revealed that it was possible to increase the average number of the sugar chain binding to one molecule of the albumin, in accordance with the amount of the sugar chain fed in the reductive amination reaction. Furthermore, it was revealed as shown in FIG. 4 that a larger number of the albumin having 20 GM1 binding per molecule on average bound to CTB, a molecule capable of recognizing the sugar chain (GM1), than the albumin having seven GM1 binding on average.

<Preparation of Membrane Support on which Protein Having Sugar Chain Binding Thereto was Immobilized>

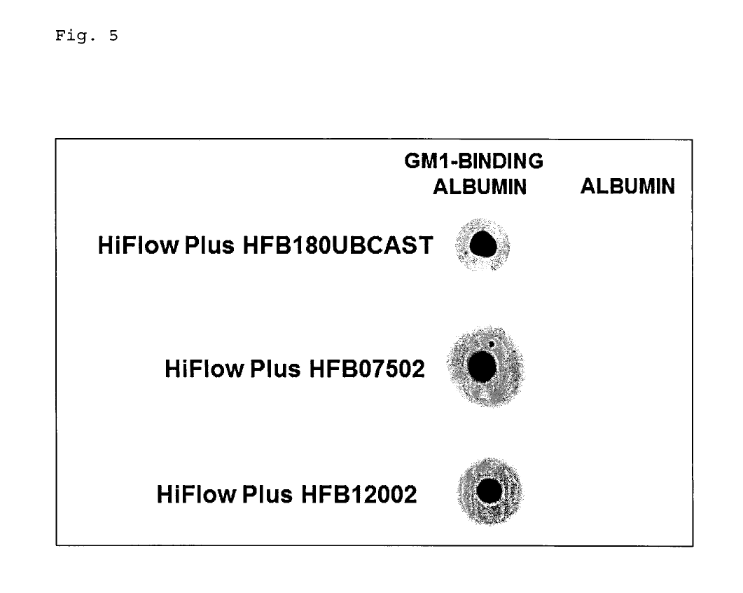

The GM1-binding albumin (the average number of the sugar chain binding to one molecule of the albumin: 20) prepared as described above or albumin, each 1 μg, was spotted on three types of immunochromatographic membranes. The membranes used were: HiFlow Plus HFB180UBCAST, HiFlow Plus HFB07502, and HiFlow Plus HFB12002 membranes made of nitrocellulose and manufactured by Millipore Corporation. Thereafter, the membranes on which the GM1-binding albumin or the albumin was spotted were dried with air, and then colored using HRP-CTB (the HRP-CTB concentration: 0.2 μg/mL) and 4-chloro-1-naphthol. FIG. 5 shows the obtained result.

As apparent from the result shown in FIG. 5, the coloring was observed at the site where the GM1-binding albumin was spotted. On the other hand, no coloring was observed at the site where only the albumin was spotted. This revealed that the immobilization was possible on all of the three immunochromatographic membranes on which the GM1-binding albumins were tested.

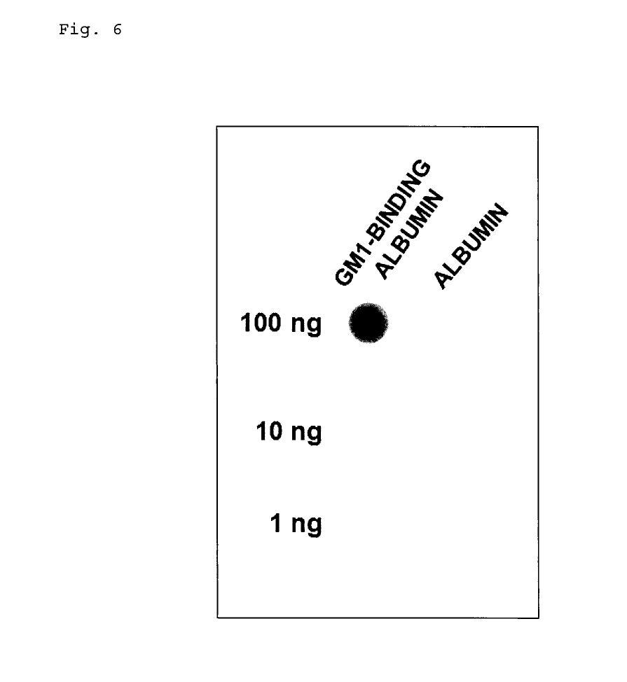

Moreover, the GM1-binding albumin was immobilized on the immunochromatographic membrane (HiFlow Plus HFB180UBCAST) as above, but the amount spotted on the membrane was changed from 1 μg to 100 ng, 10 ng, or 1 ng. Then, the detection was performed with HRP-CTB (the HRP-CTB concentration: 0.2 μg/mL) and 4-chloro-1-naphthol. FIG. 6 shows the obtained result.

As shown in FIG. 6, it was revealed that it was possible to detect the cholera toxin β subunit (hereinafter may also be referred to as “CTB”) molecule capable of binding to GM1, using the immunochromatographic membrane on which 10 ng or more of the GM1-binding albumin was immobilized.

<Verification of Membrane Support on which Protein Having Sugar Chain Binding Thereto was immobilized>

The method for detecting a glycolipid-recognizing molecule includes an ELISA method. When a glycolipid-recognizing molecule is detected by the ELISA method, a microplate is used on which 100 ng of GM1 ganglioside is normally immobilized per well (see PTL 1). Hence, such an ELISA method was compared with the immunochromatography using the membrane support of the present invention in terms of detecting a glycolipid-recognizing molecule.

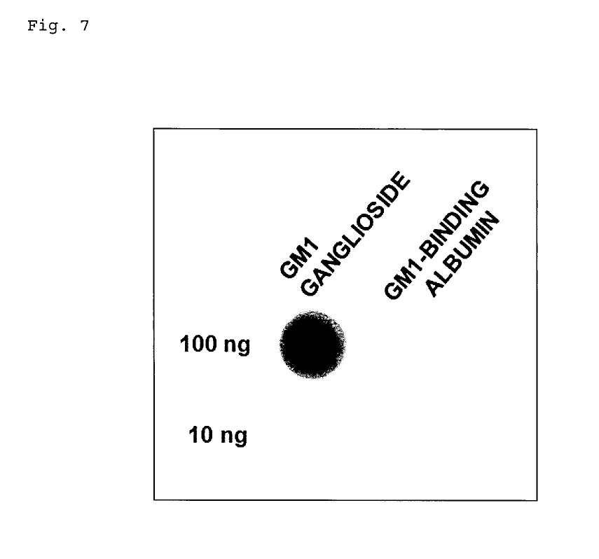

Specifically, first, membrane supports of the present invention (Examples 1 and 2) were prepared. For this preparation, 100 ng and 10 ng of the GM1-binding albumin (the average number of the sugar chain binding to one molecule of the albumin: 20) were spotted and immobilized on the membranes (HiFlow Plus HFB180UBCAST), respectively. Moreover, membrane supports (Comparative Examples 1 and 2) were prepared by immobilizing 100 ng and 10 ng of GM1 ganglioside in place of the GM1-binding albumin. Note that 100 ng and 10 ng of the GM1-binding albumin presumably contain GM1 in amounts equivalent to 50 ng and 5 ng of GM1 ganglioside, respectively. Further, HRP-CTB (the HRP-CTB concentration: 0.2 μg/mL) was allowed to act on these membrane supports, followed by staining using 4-chloro-1-naphthol as a chromogenic substrate. FIG. 7 shows the obtained result.

As apparent from the result shown in FIG. 7, the stainability of the membrane support of the present invention (Example 2) on which the GM1-binding albumin equivalent to 5 ng of GM1 ganglioside was immobilized was equal to that of the membrane support (Comparative Example 1) on which 100 ng of GM1 ganglioside was immobilized. Further, the stainability of the membrane support of the present invention (Example 1) on which the GM1-binding albumin equivalent to 50 ng of GM1 ganglioside was immobilized was remarkably superior to that of Comparative Example 1. Thus, it was revealed that the GM1-binding albumin had a remarkably higher binding ability to CTB than the GM1 ganglioside, and that the immunochromatography using the membrane support of the present invention was remarkably superior to the ELISA method using the GM1 ganglioside in detecting the glycolipid-recognizing molecule (CTB).

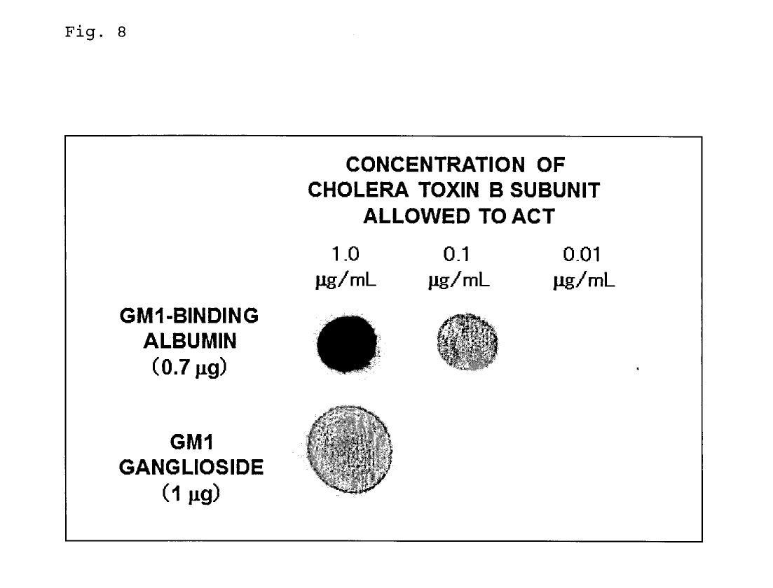

Next, verified was whether or not the GM1-binding albumin was capable of detecting the glycolipid-recognizing molecule with a favorable sensitivity. Specifically, prepared were: a membrane support of the present invention (Example 3) on which 0.7 μg of the GM1-binding albumin was immobilized as in Examples 1 and 2; and a membrane support (Comparative Example 3) on which 1 μg of GM1 ganglioside was immobilized as in Comparative Examples 1 and 2. Further, HRP-CTB was allowed to act on these membrane supports, followed by staining using 4-chloro-1-naphthol as a chromogenic substrate. Note that the HRP-CTB concentration in the HRP-CTB solution allowed to act was 1.0, 0.1, or 0.01 μg/mL. Moreover, 0.7 μg of the GM1-binding albumin presumably contains GM1 in an amount equivalent to 0.35 μg of GM1 ganglioside. FIG. 8 shows the obtained result.

As apparent from the result shown in FIG. 8, it was revealed that the membrane support (Comparative Example 3) on which 1 μg of GM1 ganglioside was immobilized was not capable of detection unless the HRP-CTB concentration was approximately 0.1 to 1.0 μg/mL; meanwhile, although the amount of the sugar chain in the GM1-binding albumin was merely approximately 35% of GM1 ganglioside, the GM1-binding albumin was capable of detection even with the HRP-CTB concentration being approximately 0.01 μg/mL.

Thus, it was revealed that immobilizing a protein having a sugar chain binding thereto on a membrane support made it possible to immobilize the sugar chain highly densely on the membrane support. Moreover, it was revealed that the use of such a membrane support made it possible to detect a sugar chain-recognizing molecule with higher sensitivity than a conventional method (for example, ELISA method).

### INDUSTRIAL APPLICABILITY

As has been described above, the present invention makes it possible to detect a sugar chain-recognizing molecule quickly and easily with high sensitivity in any place.

Thus, the immunochromatography using the membrane support of the present invention and so forth is useful in research and development in which detecting a molecule capable of recognizing a sugar chain (lectin, antibody capable of recognizing a sugar chain) is required, and in diagnosis of diseases in which an autoantibody recognizing a sugar chain is involved.

### REFERENCE SIGNS LIST

1: membrane support
2: detection line
3: control line
4: sample pad
5: conjugate pad
6: absorbent pad
7: introduction port
8: determination window
9: air hole
10: upper casing
11: lower casing
12: retaining plate
13: protrusion

1. A membrane support, comprising:
a support body having a detection line on which a protein having a sugar chain binding thereto is immobilized to detect by immunochromatography a molecule capable of recognizing a sugar chain.

2. The membrane support according to claim 1, wherein the sugar chain has a reduction terminal bound to an amino group of the protein by a reductive amination reaction.

3. The membrane support according to claim 1, wherein the protein is albumin.

4. A method for detecting by immunochromatography a molecule capable of recognizing a sugar chain, comprising:
bringing a test sample into contact with a molecule which is labeled and recognizes a molecule capable of recognizing a sugar chain;
developing the test sample contacted with the molecule which is labeled toward the detection line of the membrane support according to claim 1; and
detecting a labeled substance on the detection line.

5. A device for detecting by immunochromatography a molecule capable of recognizing a sugar chain, the device comprising:
the membrane support according to claim 1; and
at least one of a sample pad, a conjugate pad, and an absorbent pad.

6. The device according to claim 5, wherein the sample pad is disposed on an upstream side of the membrane support.

7. The device according to claim 5, wherein the conjugate pad is disposed on an upstream side of the membrane support.

8. The device according to claim 5, wherein the absorbent pad is disposed on a downstream side of the membrane support.

9. The membrane support according to claim 1, wherein the protein is immobilized on the detection line in an amount of from 1 ng to 1 μg.

10. The membrane support according to claim 1, wherein the protein is immobilized on the detection line via physical absorption, electrostatic interaction, hydrophobic interaction, or a crosslinker.

11. The membrane support according to claim 1, wherein the membrane support comprises at least one material selected from the group consisting of nitrocellulose, polyvinylidene fluoride, polyethersulfone, polyethylene terephthalate, polyethylene, polypropylene, nylon, and a glass fiber.

12. The membrane support according to claim 1, wherein the sugar chain bound to the protein is a sugar chain of a glycolipid.

13. The membrane support according to claim 12, wherein the glycolipid is ganglioside GM1.

14. The membrane support according to claim 2, wherein the protein is albumin.
